(19) United States
(12) Patent Application Publication
(10) Pub. No.: US 2019/0046169 A1
(43) Pub. Date: Feb. 14, 2019

(54) MULTI-FUNCTIONAL OCCLUDER

(71) Applicant: NAGESWARA RAO KONETI, HYDERABAD (IN)

(72) Inventor: NAGESWARA RAO KONETI, HYDERABAD (IN)

(21) Appl. No.: 15/759,832

(22) PCT Filed: Aug. 2, 2016
(86) PCT No.: PCT/IN16/00201

§ 371 Date: Mar. 14, 2018

(30) Foreign Application Priority Data
Sep. 18, 2015 (IN) 5012/CHE/2015

(51) Int. Cl. A61B 17/00; A61B 17/12

(52) U.S. Cl. CPC A61B 17/0057; A61B 2017/00867; A61B 2017/00592; A61B 17/12022

(57) ABSTRACT

The present invention is related to an occlusion device for occluding an opening in a body tissue and a method of deploying the said occlusion device to the site of defect. The construction of the occlusion device comprising two discs, that are centrally connected by a central portion and retention screws, is such that it offers the major advantage of haemodynamic adjustment providing a better-fit to the size of the defect.

### CROSS-REFERENCE TO RELATED APPLICATIONS

The present application is a continuation of an International Application Number PCT/IN2016/000201, with a filing date of Aug. 2, 2016, the entire disclosure of which is incorporated herein by reference for all purposes. The present application claims the benefit of foreign priority application number 5012/CHE/2015, with a filing date of Sep. 18 2015, the entire disclosure of which is incorporated herein by reference for all purposes.

### FIELD OF INVENTION

The present invention is generally related to an intravascular occlusion device for the treatment of certain medical conditions and, more particularly, related to a multi-functional occlusion device for the treatment of congenital defects.

### BACKGROUND OF THE INVENTION

A wide variety of intravascular devices are used in various medical procedures. Certain intravascular devices, such as catheters and guidewires, are generally used to just deliver fluids or other medical devices to specific locations within a patient's body, such as a selective site within the vascular system. Other, frequently more complex, devices are used in the treatment of specific conditions, such as devices used in the removal of vascular occlusions or for the treatment of septal defects and the like.

Congenital heart diseases include patent foramen ovale (PFO), atrial septal defect (ASD), patent ductus arteriosus (PDA), ventricular septal defect (VSD), etc. PFO and ASD are openings in the wall between the right atrium and left atrium of the heart thereby creating the possibility of the passage of blood from the right atrium to the left atrium. But the defect size of PFO is usually smaller than that of ASD and the defect does not extend perpendicularly to the septal wall, i.e. the left atrial septal defect is not concentric with that of the right atrium. Once the occluder has been placed, it prevents the thrombus from entering the left atrium. Furthermore, the atrial septal defect (ASD) is usually larger and requires repair. Currently, there are many types of endocardiac occlusion devices for the treatment of congenital heart diseases. These occluders are delivered to the desired location using a corresponding catheter.

Several devices such as the Amplatzer Septal Occluder, Gore Helex Septal Occluder and Occlutech Figula have been developed to occlude these defects. The Amplatzer design, disclosed in U.S. Pat. No. 5,725,552, U.S. Pat. No. 5,846,261, U.S. Pat. No. 5,944,738 and U.S. Pat. No. 6,123,715, has braided and woven nitinol wires that are shape set into two discs with a thinner middle portion, such that the middle portion is placed through the opening and the two discs clamp down on each side of the body tissue.

In the past, mechanical occlusion devices have been proposed for the treatment of congenital heart diseases, some of which have an accurate diameter of the defect to be closed while in certain cases, it may not always be correct in all clinical situations such as Perimembranous Ventricular septal defect (Pm VSD), para-valvular leak and Coronary arterio-venous fistula (CAVF). The physician tends to use an over-sized device to prevent embolization or residual leak. This over-sizing may lead to complications such as heart blocks, distortion of the device and damage to the intra- or extra-cardiac structures. However, these devices may be difficult to adapt to a variety of short and long tunnel widths.

Prior to implantation of these devices, it is important to determine the thickness of the septal wall near the defect and the approximate width of the defect, in order to provide an appropriately sized device. A balloon catheter and a calibrated guidewire having radiopaque regions of known length, may be utilized by a physician during a preliminary fluoroscopic procedure to estimate the defect's size, shape and thickness of the septal wall near the defect. Although useful, the exact size and shape of the defects cannot be determined, thereby increasing the possibility of leakage around the occluding device. Hence, a device that inherently adjusts to the shape and thickness of the defect would be desirable.

Further, the shapes (for example squares, triangles, pentagons, hexagons and octagons) of the devices in the prior art require a larger surface contact area and have corners which may extend to the free wall of the atria. Each time the atria contracts (approximately 100,000 times per day), the corners extending to the atria walls are bent, thus creating structural fatigue fractures in approximately 30 percent of all cases. Furthermore, the previous devices require a French 14-16 introducing catheter, making it impossible to treat children affected with congenital defects using these devices. Hence, it would be advantageous to provide a reliable embolization device which is both easy to deploy through a 6-7 French catheter and which automatically adjusts to the shape and thickness of the defect.

CN 1106828, CN 1091584 and CN 1102373 disclose mechanical occlusion devices for the treatment of congenital heart diseases. Such devices include a support mesh with contractibility and biocompatible materials that are connected to the circumference of the support mesh. The support mesh, which is first placed in the catheter, is delivered to the desired location, and is then deployed to close the septal defect. Such devices are easy to withdraw and have excellent centricity. However, the left disc of such devices directly contact the blood, and can potentially form a thrombus and release harmful metallic elements more easily. Moreover, as the two discs are integrated, they cannot automatically adjust angularly to adapt to the unique anatomy of the patient. Meanwhile, if the left disc is not deployed completely, the operation becomes more complicated.

In addition, with the existing techniques and operation methods, it is very difficult to determine the size and shape of the septal defect precisely, and with the limiting waist size, difficulties such as selection error are encountered by the physicians. If an oversized device is selected, the occluder will form a cucurbit shape and result in an imperfect closing effect.

Considering the above stated difficulties associated with occlusion procedures and devices available in the prior art, there is a strong need for devices that are more effective and can provide a constant inward axial pressure on each side of the body tissue. Although some of these devices are adequate, there is a need for devices that provide a more effective fit and grip for a variety of openings.

The devices disclosed herein are designed to address these and other drawbacks of prior art septal closure devices.

### OBJECT OF THE INVENTION

It is accordingly a principal object of the present invention to provide a device suitable for occluding various septal defects.

Another object of the present invention is to provide an occluding device that has multi-functionality and can be applicable for various defects.

Yet another object of the invention is to provide an occluder with a haemodynamic advantage to adjust according to the size of the defect thereby providing an excellent fit.

These and other objects, of the present invention will become readily apparent to those skilled in the art from a review of the following detailed description of the preferred embodiment in conjunction with the accompanying claims and drawings in which numerals in the several views refer to corresponding parts.

### SUMMARY OF THE INVENTION

The present invention is related to an intravascular occlusion device for the treatment of certain medical conditions and more particularly, related to a multi-functional occlusion device for the treatment of congenital defects such as Atrial and Ventricular Septal Defects (ASD and VSD respectively), Perimembranous Ventricular septal defect (Pm VSD), Pm VSD with membranous septal aneurysm, Muscular VSD, Post-operative residual VSD, Coronary arterio-venous fistula (CAVF), Systemic arterio-venous fistula (SAVF), Systemic to pulmonary collaterals, Rupture of sinus of Valsalva (RSOV), selected cases of aorto-pulmonary window as well as conditions that result from previous medical procedures such as Para-Valvular Leaks (PVL) following surgical valve repair or replacement. More particularly, the device has the ability to adjust haemodynamically as per the clinical situation.

The present invention is related to an occlusion device comprising of two discs: a high-pressure disc and a low-pressure disc; connecting portion; and a plurality of retention screws. The said high-pressure disc and the said low-pressure disc are held in place using retention screws. The said retention screws are located on either side of the discs. The said two discs of the occlusion device are held together through a central connection portion. The conical connection section lends a hemodynamic advantage.

The present invention is also related to a method for occluding an opening in a body tissue by deploying an occlusion device to the required site. The method comprises the deployment of the occlusion device through the trans-venous approach or trans-arterial approach also known as antegrade and retrograde methods.

Further features and advantages of the present disclosure will become apparent from consideration of the following description and the appended claims when taken in connection with the accompanying drawings. It should be understood that the description and specific examples are intended for purposes of illustration only and are not intended to limit the scope of the present disclosure.

### BRIEF DESCRIPTION OF THE DRAWINGS

FIG. 1 is a human heart with a ventricular septal defect wherein the present invention is used in accordance with some embodiments of the present disclosure.

FIG. 2 is a perspective view of an occlusion device with two discs—a high-pressure disc and low-pressure disc; FIG. 2(a) illustrates the basic design of the present invention.

FIG. 3 is a perspective view of an occlusion device which comprises two disc-shaped portions along with retention screws.

FIG. 4 illustrates the central disc as a conical structure connecting the high-pressure disc and low-pressure disc.

FIG. 5 illustrates the method of deployment of the occlusion device in the patient body through antegrade approach.

FIG. 6 illustrates the method of deployment of the occlusion device in the patient body through retrograde approach.

### DETAILED DESCRIPTION

The present invention is related to devices intended to occlude an aperture within a body tissue. The term “occlusion device” in the present disclosure is meant for the interpretation of the device for occluding a defect in a living body. In particular and as described in detail below, the described multifunctional occlusion device may be used for closing various septal defects in the heart. The occlusion device comprises of two discs; a high-pressure disc and a low-pressure disc that are held in place using retention screws on either side. The said two discs are further interconnected through a central connection which according to some embodiments may be a conical structure.

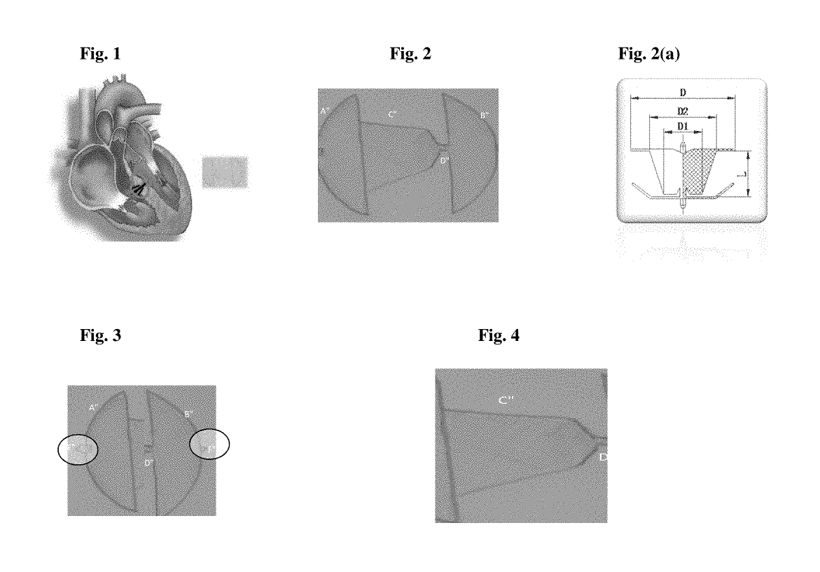

FIG. 1 illustrates a human heart having a right atrium, a left atrium and a ventricular septal defect.

FIG. 2 illustrates the occlusion device for use in occluding an abnormal opening in a patient's body. In an embodiment, the occlusion device comprises of two disc-shaped portions ‘A’ & ‘B’ connected through a central connection portion ‘C’. The said discs are high-pressure disc and low-pressure disc. The said discs can be deployed on either side depending on the clinical situation.

FIG. 2 (a) further illustrates the basic design of the device, wherein “D” represents the overall diameter of the device, “D1” represents the diameter at the tapered lower end, and “D2” represents the diameter at the maximum end. “L” represents the length of the device.

FIG. 3 illustrates the occlusion device comprising of two disc-shaped portions with two retention screws ‘E’ & ‘F’. The said retention screws present on either side of the discs aid in the retention of the discs in place after the deployment on the side of choice, which depends on the clinical situation.

FIG. 4 illustrates the connectivity between the high-pressure disc ‘A’ and low-pressure disc ‘B’ through a central disc ‘C’. The central disc presented in FIG. 2(a) is a conical structure with varying diameters with the diameter decreasing from ‘D’ to ‘D2’ to ‘D1’. The conical structure determines the size of the device. The length of the conical structure is variable depending on the situation, and can be customized. The diameter of the conical structure can range, but not limiting to, for example, from a maximum of 7/5 device at the broader end to a minimum of 7 mm further tapering to 5 mm at “D”.

According to an embodiment, the occluder of the present invention has the ability to be adjusted by the high-pressure chamber as per the required diameter at the site of defect. The determination of the diameter of the defect that is to be closed may not always be accurate in all clinical situations, particularly in situations such as Pm VSD, para-valvular leak and CAVF. The physician tends to use over-sized device to prevent embolization or residual leak. This may lead to oversizing related complications such as heart blocks, distortion of device and damage to intra- or extra-cardiac structures. This is solved by the said conical structure of the present invention that lends a haemodynamic advantage to adjust according to the size of the defect.

According to a preferred embodiment, this hemodynamic advantage enables the placement of the high-pressure disc in the high-pressure chamber and the low-pressure disc in the low pressure chamber, based on the clinical situation.

The disc ‘C’ can be adjusted as per the diameter of the defect after positioning within the desired position to close. The high pressure in the cardiac chamber pushes the device to the desired diameter and adjusts according to the diameter. This haemodynamic advantage aligns the device properly within the defect and prevents over sizing thus complications related to that.

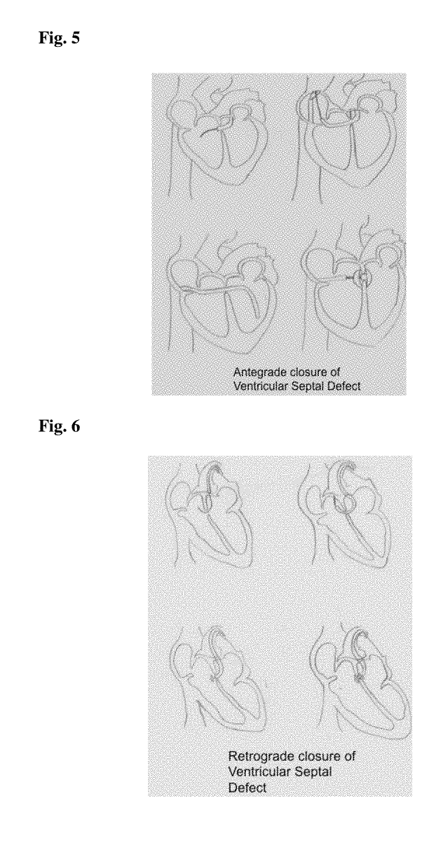

FIG. 5 illustrates a method of deploying the occlusion device of the present invention in a patient's body. The device can be deployed employing the trans-venous approach called as antegrade method. The antegrade approach involves forming an arterio-venous looping and applicable for the closure of defects such as the left to right shunts, fistulae and para-valvular leaks.

FIG. 6 illustrates a method of deploying the occlusion device of the present invention in a patient's body. The device can be deployed employing a retrograde approach (trans-arterial approach). The retrograde approach is simple and applicable for the closure of defects such as Pm VSDs, CAVF, SAVF and other variety of conditions.

According to an embodiment, the device can either have a membrane or not have a membrane. According to yet another embodiment, the membrane can be a PTFE membrane. According to a preferred embodiment, there is no membrane for the sizes 5/3-8/6, whereas a PTFE membrane is used for the sizes 9/7-14/12.

Table 1 illustrates the basic design and the variations in size, which are only illustrative and non-limiting examples. Modifications can be made as per the clinical situation and be customized.

TABLE 1
Design and variables of a multi-functional occluder

| | | | | | Recommended | |
|---|---|---|---|---|---|---|
| Size | D | D1 | D2 | L | Sheath | Membrane |
| LT-5-3 | 10 | 3 | 5 | 4 | 5F | No |
| LT-6-4 | 10 | 4 | 6 | 4 | 5F | No |
| LT-7-5 | 12 | 5 | 7 | 4 | 5F-6F | No |
| LT-8-6 | 12 | 6 | 8 | 4 | 5F-6F | No |
| LT-9-7 | 14 | 7 | 9 | 4 | 7F | Yes |
| LT-10-8 | 14 | 8 | 10 | 4 | 7F | Yes |
| LT-12-10 | 16 | 10 | 12 | 4 | 7F | Yes |
| LT-14-12 | 18 | 12 | 14 | 4 | 7F | Yes |

The aforementioned table lists the various sizes of the device and diameter, according to some of the embodiments of the present invention.

### Example 1

The LT-5/3 device has 5 mm as the maximum diameter at “D2” and 3 mm minimum diameter at the tapering end “D1”. The length of the device is 4 mm and the recommended sheath for the device is 5 F. There is no membrane present for this size.

### Example 2

The LT-6/4 device has 6 mm maximum diameter at “D2” and 4 mm minimum diameter at lower end “D1”. The length of the device is 4 mm and the recommended sheath for the device is 5 F. There is no membrane present for this size.

### Example 3

The LT-7/5 device has 7 mm maximum diameter at “D2” and 5 mm minimum diameter at lower end “D1”. The length of the device is 4 mm and the recommended sheath for the device is 5 F-6 F. There is no membrane present for this size.

### Example 4

The LT-8/6 device has 8 mm maximum diameter at “D2” and 6 mm minimum diameter at lower end “D1”. The length of the device is 4 mm and the recommended sheath for the device is 5 F-6 F. There is no membrane present for this size.

### Example 5

The LT-9/7 device has 9 mm maximum diameter at “D2” and 7 mm minimum diameter at lower end “D1”. The length of the device is 4 mm and the recommended sheath for the device is 7 F. PTFE membrane is present for this size.

### Example 6

The LT-10/8 device has 10 mm maximum diameter at “D2” and 8 mm minimum diameter at lower end “D1”. The length of the device is 4 mm and the recommended sheath for the device is 7 F. PTFE membrane is present for this size.

### Example 7

The LT-12/10 device has 12 mm maximum diameter at “D2” and 10 mm minimum diameter at lower end “D1”. The length of the device is 4 mm and the recommended sheath for the device is 7 F. PTFE membrane is present for this size.

### Example 8

The LT-14/12 device has 14 mm maximum diameter at “D2” and 12 mm minimum diameter at lower end “D1”. The length of the device is 4 mm and the recommended sheath for the device is 7 F. PTFE membrane is present for this size.

According to an embodiment of the present invention, a shape memory alloy suitable such as Ni—Ti available under the more commonly known name Nitinol (Nickel and Titanium alloy), may be used for the manufacture of the occlusion device. The standard technique for manufacturing the device is Nitinol wire (0.0020″-0.0026″ wire) and molded.

The present invention is related to a multi-functional occluder which possesses the advantages of hemodynamic adjustment of the discs of the occlusion device to provide a better-fit, leak-proof occlusion of the defect site; customizable; and ease of handling either by a left-handed or right-handed approach.

The aforementioned described features help alleviate the problems associated with prior art technologies. It will be appreciated by those skilled in the art that various modifications and alternatives to those details could be developed in light of the overall teachings of the disclosure. Accordingly, the particular arrangements disclosed are meant to be illustrative only and not limiting as to the scope of the invention which is to be given the full breadth of the appended claims and any and all equivalents thereof.

It is to be understood, however, that the present invention would not be limited by any means to the parts, arrangements and materials that are not specifically described, and any change to the materials, variations, sizes and modifications can be made without departing from the spirits and scope described in the present invention.

1. A multi-functional occlusion device comprising of:
a flexible proximal disc preferably a high-pressure disc and a flexible distal disc preferably a low-pressure disc;
a septum;
retention screws on the outer side of said proximal and distal discs; and
a central geometrically shaped connector section;
wherein the said proximal disc and distal disc are of uniform size, and straddle the said septum to provide stability on opposite sides of a defect; and
the said central geometrically shaped connector section is cone shaped, and provides hemodynamic adjustment of the said proximal disc and distal disc.

2. The multi-functional occlusion device according to claim 1, wherein the said proximal disc and distal disc function as high-pressure disc and low-pressure disc, respectively, in the ventricular and arterial sections wherein the pressure gradient is high.

3. The multi-functional occlusion device according to claim 1, wherein the retention screws present on the outer side of the said discs enable deployment through trans-venous (antegrade) or trans-arterial (retrograde) approach.

4. The multi-functional occlusion device according to claim 1, wherein the central geometrically shaped connector section connects the said proximal disc and distal disc to maintain the high-pressure disc in the high-pressure chamber of the heart and the low-pressure disc in the low-pressure chamber of the heart, and provides hemodynamic adjustment.

5. The multi-functional occlusion device according to claims 1 and 4, wherein the central geometrically shaped connector section that connects the said proximal disc and distal disc, has a variable size and length, to enable a tight fit depending on the defect size.

6. The multi-functional occlusion device according to claim 1, wherein the device may or may not include a membrane.

7. A method for occluding an opening in a body tissue with the multi-functional occlusion device of claims 1-6, comprising of deployment of the said multi-functional occlusion device through a smaller guide catheter or delivery sheath from an inner member at the opening in the body tissue, through trans-venous (antegrade) or trans-arterial (retrograde) approach.

8. (canceled)

9. The method according to claim 7, wherein the antegrade approach of forming an arterio-venous looping can be used for the closure of defects such as left to right shunts, fistulas and para-valvular leaks.

10. The method according to claim 7, wherein the retrograde approach can be used for the closure of defects such as Pm VSDs, CAVF, SAVF and the like.
